(19) United States
(12) Patent Application Publication
(10) Pub. No.: US 2021/0204904 A1
(43) Pub. Date: Jul. 8, 2021

(54) ULTRASOUND DIAGNOSTIC SYSTEM

(71) Applicant: LILY MEDTECH INC., Tokyo (JP)

(72) Inventors: Naoki TOMII, Tokyo (JP); Hirofumi NAKAMURA, Tokyo (JP)

(21) Appl. No.: 17/210,929

(22) Filed: Mar. 24, 2021

Related U.S. Application Data
(63) Continuation of application No. PCT/JP2019/041335, filed on Oct. 21, 2019.

(30) Foreign Application Priority Data
Oct. 22, 2018 (JP) 2018-198658

(51) Int. Cl. A61B 8/08; A61B 8/14; A61B 8/00; G06N 20/00

(52) U.S. Cl. CPC A61B 8/085; G06N 20/00; A61B 8/4488; A61B 8/14

(57) ABSTRACT

An ultrasound diagnostic system includes elements that are arranged around a test object and perform at least one of emission and reception of ultrasound, a data collection unit that collects measurement data of reflected ultrasound that is ultrasound reflected from the test object through at least one of the elements while switching an element that emits ultrasound, and a first learner that learns using training data including a test-object model and simulation measurement data to output a tomographic image of the test object from the measurement data, which is input to the first learner, the test-object model being expressed by an acoustic feature distribution, the simulation measurement data being obtained by emitting ultrasound while switching an element that emits ultrasound and receiving reflected ultrasound from the test-object model using least one of the elements in a simulation space in which a size and an arrangement of the elements are simulated.

### CROSS-REFERENCE TO RELATED APPLICATIONS

This application is a Continuation of International Patent Application No. PCT/JP2019/041335, filed Oct. 21, 2019, which claims the benefit of Japanese Patent Application No. 2018-198658, filed Oct. 22, 2018, both of which are hereby incorporated by reference herein in their entirety.

### TECHNICAL FIELD

The present invention relates to an ultrasound diagnostic system in which ultrasound irradiation is performed and a tomographic image of a test object is generated.

### BACKGROUND ART

A noninvasive diagnostic system using ultrasound is widely used in the medical field as a technology for making a diagnosis based on information regarding the inside of a test object since there is no need to perform surgery in which a direct incision is made to carry out an observation in a living body.

In ultrasound computed tomography (CT), which is a technique for making a diagnosis using ultrasound, a test object is irradiated with ultrasound and a tomographic image of the test object is generated using reflected ultrasound or transmitted ultrasound. A recent study shows that ultrasound CT is useful in detecting breast cancer. In ultrasound CT, for example, a ring array transducer obtained by arranging, in a ring shape, many elements that emit and receive ultrasound is used to generate tomographic images.

For example, while switching an element that emits ultrasound in order, echo signals are received by all the elements and are stored as RF data (raw data). An image signal representing a tomographic image is then generated on the basis of the RF data.

Hitherto, when a tomographic image is reconstructed, approximation calculations such as the sound speed being treated as a constant have been performed. The amount of information of RF data is reduced through the approximation calculations, and thus it becomes difficult to form a clear tomographic image, resulting in an impediment to improving the accuracy with which a diagnosis is made.

International Publication No. 2017/051903 is an example of related art.

### SUMMARY OF INVENTION

The present invention has been made in light of the existing circumstances described above, and an object of the present invention is to provide an ultrasound diagnostic system that generates a clear, accurate ultrasound image.

An ultrasound diagnostic system according to one aspect of the present invention includes a plurality of elements that perform at least one of emission of ultrasound to a test object and reception of reflected ultrasound that is ultrasound reflected from the test object, a data collection unit that collects measurement data of the reflected ultrasound through at least one of the plurality of elements while switching an element that emits ultrasound, and a first learner that learns using training data including a test-object model and simulation measurement data to output a tomographic image of the test object from the measurement data, which is input to the first learner, the test-object model being expressed by a distribution of acoustic features, the simulation measurement data being obtained by emitting ultrasound while switching an element that emits ultrasound and receiving reflected ultrasound from the test-object model using at least one of the plurality of elements in a simulation space in which a size and an arrangement of the plurality of elements are simulated.

An ultrasound diagnostic system according to another aspect of the present invention includes a plurality of elements that perform at least one of emission of ultrasound to a test object and reception of reflected ultrasound that is ultrasound reflected from the test object, a data collection unit that collects measurement data of the reflected ultrasound through at least one of the plurality of elements while switching an element that emits ultrasound, and a first learner that learns using training data including a brightness image and simulation measurement data to output a tomographic image of the test object from the measurement data, which is input to the first learner, the brightness image being based on a natural image, the simulation measurement data being obtained by emitting ultrasound while switching an element that emits ultrasound and receiving reflected ultrasound from an acoustic feature distribution based on the brightness image using at least one of the plurality of elements in a simulation space in which a size and an arrangement of the plurality of elements are simulated.

The ultrasound diagnostic system may further include an image acquisition unit that inputs the measurement data collected by the data collection unit to the first learner and acquires a tomographic image output from the first learner.

The ultrasound diagnostic system may further include a second learner capable of determining a presence or absence of and a position of a tumor in a tomographic image of a test object through learning using training data including a training tomographic image and tumor information determining a position of a tumor included in the training tomographic image, and a determination unit that inputs the tomographic image acquired by the image acquisition unit to the second learner and outputs, on a basis of output data from the second learner, a determination result on a presence or absence of a tumor in the tomographic image and, in a case where a tumor exists, information on a position of the tumor.

The plurality of elements may be arranged around the test object.

Further features of the present invention will become apparent from the following description of exemplary embodiments with reference to the attached drawings.

### BRIEF DESCRIPTION OF DRAWINGS

FIG. 1 is a schematic diagram of the configuration of an ultrasound diagnostic system according to an embodiment of the present invention;

FIG. 2 is a section view taken along line II-II of FIG. 1;

FIG. 3 is a functional block diagram of a calculation device;

FIG. 4 is a schematic diagram of measurement data;

FIG. 5 is a schematic diagram of propagation of ultrasound;

FIG. 6 is a diagram illustrating partial RF data of a point of interest;

FIG. 7 is a schematic diagram of the structure of a learner;

FIG. 8 is a schematic diagram illustrating an image reconstruction method;

FIG. 9A illustrates a biological model, FIG. 9B illustrates a measurement image, and FIG. 9C is a diagram illustrating an image reconstructed from an output from the learner;

FIG. 10 is a diagram illustrating an example of emission and reception at a plurality of elements;

FIG. 11 is a diagram illustrating an example of partial RF data;

FIGS. 12A and 12B are diagrams illustrating examples of propagation paths of ultrasound, and FIG. 12C is a diagram illustrating partial RF data;

FIG. 13 is a diagram illustrating an example in which a brightness image is converted into a density distribution; and

FIG. 14A illustrates a brightness image, FIG. 14B illustrates a density distribution image, and FIG. 14C illustrates a reconstructed image.

### DESCRIPTION OF EMBODIMENTS

In the following, embodiments of the present invention will be described in more detail with reference to the drawings. An ultrasound diagnostic system according to an embodiment of the present invention irradiates a test object such as a human body with ultrasound and generates a tomographic image (an ultrasound image) using received reflected-wave signals. A doctor makes a diagnosis by checking the generated tomographic image.

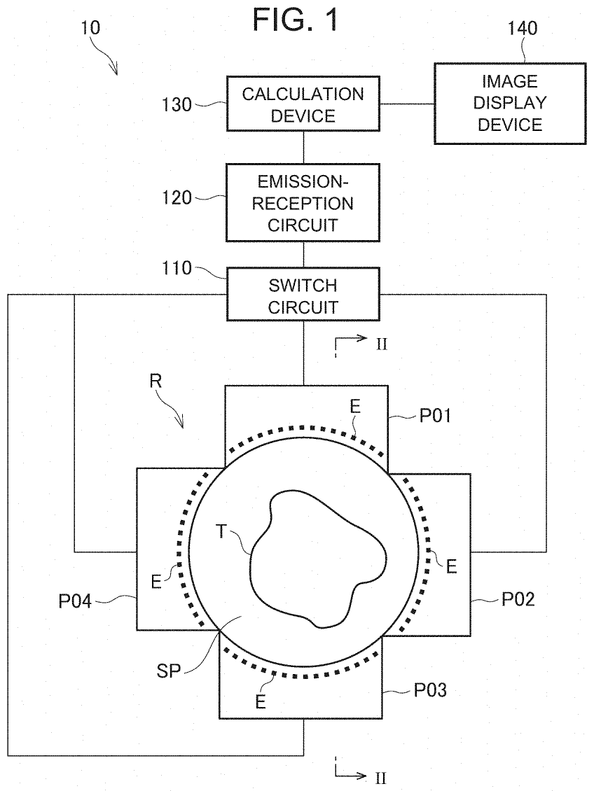

As illustrated in FIG. 1, an ultrasound diagnostic system 10 according to the present embodiment includes a ring array R, a switch circuit 110, an emission-reception circuit 120, a calculation device 130, and an image display device 140.

The ring array R is a ring-shaped transducer constituted by a combination of a plurality of transducers and having preferably a diameter of 80 to 500 mm and more preferably a diameter of 100 to 300 mm. The ring array R may have a variable diameter. In the present embodiment, as an example, a ring-shaped transducer obtained by combining four concave transducers P01 to P04 is used.

For example, in a case where the concave transducers P01 to P04 each have 512 rectangular piezoelectric elements E (hereinafter also simply referred to as “elements E”), the ring array R is constituted by 2048 elements E. The number of elements E provided at the concave transducers P01 to P04 is not limited to a specific number and is preferably between 1 and 1000.

Each element E has the function of converting an electrical signal into an ultrasonic signal and converting an ultrasonic signal into an electrical signal. The element E emits ultrasound to a test object T, receives reflected waves that are waves reflected by the test object T, and forms an electrical signal as measurement data.

In the present embodiment, each element E is described as an element having the function of both emitting and receiving ultrasound; however, the element E is not limited to this. For example, emission elements or reception elements may be used, which have either one of the function of emitting ultrasound and the function of receiving ultrasound, and a plurality of emission elements and a plurality of reception elements may be arranged in a ring shape. In addition, the ring array R may be constituted by the element (or elements) having the function of both emitting and receiving ultrasound, the emission element (or elements), and the reception element (or elements) in a mixed manner.

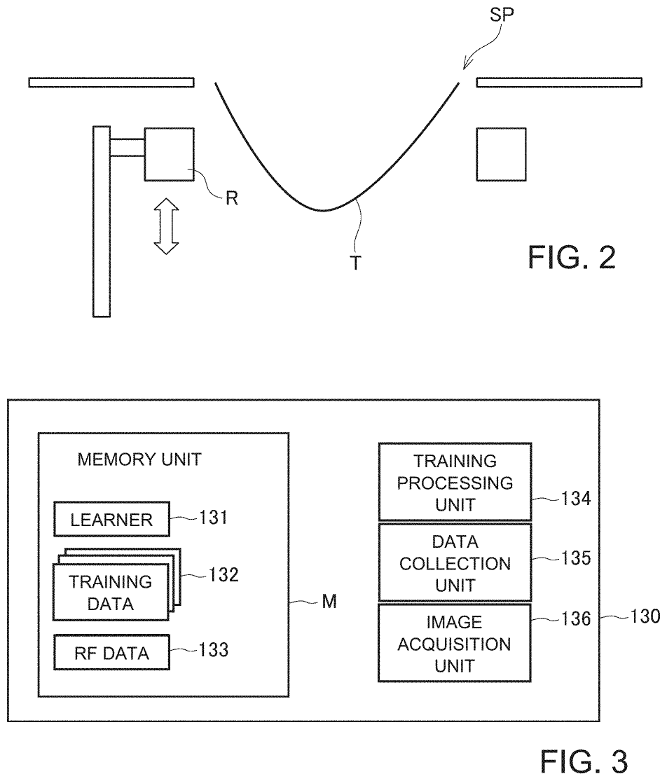

FIG. 2 is a section view taken along line II-II of FIG. 1. For example, the ring array R is installed under a bed having an opening such that the opening of the bed is superposed with an insertion portion SP. A test subject inserts a site of his or her body to be imaged (the test object T) into the insertion portion SP from the opening of the bed.

The insertion portion SP, into which the test object T is inserted, is provided at the center of the ring array R. The plurality of elements E of the ring array R are provided at equal intervals along the ring around the insertion portion SP. Convex lenses called acoustic lenses are attached to the inner peripheral side surface of the ring array R. Such surface treatment added on the inner peripheral side of the ring array R can cause ultrasound emitted by each element E to converge within a plane including the ring array R.

In the present embodiment, the elements E are arranged in a ring shape and at equal intervals; however, the shape of the ring array R is not limited to a circular shape and may be, for example, an arbitrary polygonal shape such as a hexagon, a square, and a triangle, a shape having at least partially a curve or an arc, another arbitrary shape, or a portion of these shapes (for example, a semicircle or an arc). That is, the ring array R can be generalized as an array R. In addition, the elements E constituting the array R are preferably arranged intermittently around the test object T so as to cover 90 degrees or more; however, the arrangement of the elements E is not limited to these.

The ring array R is connected to the emission-reception circuit 120 with the switch circuit 110 interposed therebetween. The emission-reception circuit 120 (control unit) transmits a control signal (electrical signal) to the elements E of the ring array R and controls emission and reception of ultrasound. For example, the emission-reception circuit 120 sends, to the elements E, a command as to for example the frequency and magnitude of ultrasound to be emitted and the type of wave (such as a continuous wave or a pulse wave).

The switch circuit 110 is connected to each of the plurality of elements E of the ring array R, transfers a signal from the emission-reception circuit 120 to certain elements E among the plurality of elements E, and drives the elements E to emit-receive a signal. For example, by switching the elements E to which the control signal from the emission-reception circuit 120 is supplied, the switch circuit 110 causes one of the plurality of elements E to function as an emission element, which emits ultrasound, and causes a plurality of elements E (for example, all the elements E) to receive reflected waves.

The ring array R is installed so as to be movable up and down by, for example, a stepping motor. Data on the entirety of the test object T can be collected by moving the ring array R up and down.

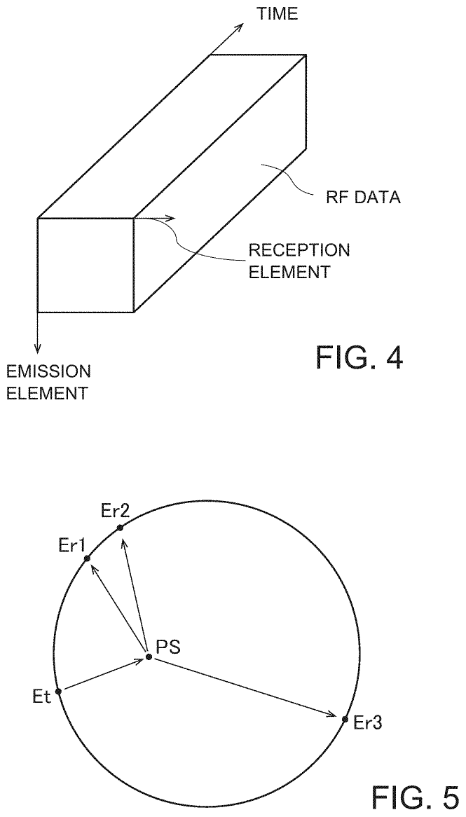

Next, measurement data (RF data), which is data obtained by a plurality of elements E, will be described. Ultrasound emitted from one emission element is reflected by the test object T and received by a plurality of reception elements. As a result, two-dimensional RF data is obtained in which a first axis represents reception-element number and a second axis represents reflected-wave propagation time. Pieces of two-dimensional data, the number of which is equal to the number of emission elements, is obtained by performing measurement while switching an emission element. In other words, three-dimensional RF data as illustrated in FIG. 4 is obtained in which a first axis represents reception-element number, a second axis represents reflected-wave propagation time, and a third axis represents emission-element number.

FIG. 5 is a schematic diagram illustrating the way in which ultrasound emitted from one emission element Et is reflected (scattered) by one point scatterer PS (one point on the test object T) and is received by a plurality of reception elements Er1, Er2, and Er3. Since ultrasound propagation paths (propagation distances) differ from each other, the reflected-wave propagation times to the respective reception elements differ from each other.

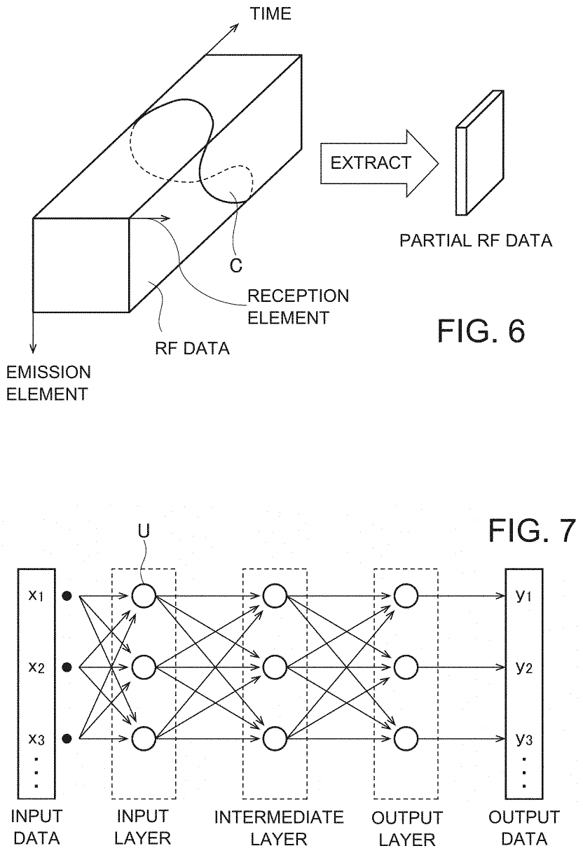

Thus, the waves reflected by the point scatterer PS and measured at the respective reception elements form a curve in two-dimensional RF data, in which the first axis represents reception-element number and the second axis represents reflected-wave propagation time. Moreover, the waves reflected by the point scatterer PS and measured at the respective reception elements are distributed on a curved surface C as illustrated in FIG. 6 in three-dimensional RF data. In the present embodiment, partial RF data corresponding to the curved surface C is extracted, the partial RF data is input to a trained learner, and a partially reconstructed image corresponding to the point scatterer PS is output.

As illustrated in FIG. 3, the calculation device 130 is constituted by, for example, a computer including a central processing unit (CPU), a communication unit, and a memory unit M. The memory unit M has, for example, a random-access memory (RAM), a read-only memory (ROM), and a hard disk. The functions of, for example, a data collection unit 135 and an image acquisition unit 136 are realized by executing an image reconstruction program stored in the memory unit M, and a measurement data storage area 133 is reserved in the memory unit M. Processing performed by each unit will be described later.

A learner 131 is stored in the memory unit M. The learner 131 is a processing execution program for performing processing on parameters such as weights and a bias regarding each unit (neuron) as well as input data. “A learner 131 is stored in the memory unit M” means that various parameters and the processing execution program regarding the learner 131 are stored in the memory unit M.

A training processing unit 134 executes training processing for the learner 131 using training data 132 stored in the memory unit M. The training data 132 includes RF data from a simulation regarding a biological model (test-object model) and a measurement image of the biological model (an ideal measurement image calculated from acoustic features of the biological model such as the strength of spatial gradient of acoustic impedance), the biological model being expressed by a distribution of acoustic features. In the simulation, the size and arrangement of the plurality of elements E of the ring array R are simulated in a simulation space and reflected ultrasound from the biological model is received by a plurality of elements while switching an emission element. A plurality of sets of simulation RF data and a measurement image are prepared.

The simulation RF data is input to the learner 131, and the learner 131 is trained such that output data from the learner 131 matches pixel values of the measurement image.

The learner 131 uses a neural network. FIG. 7 is a diagram illustrating the structure of the learner 131. Input data for the learner 131 includes a plurality of input variables x₁, x₂, x₃, . . . . The input variables of each input data are values of reception signals of the respective reception elements included in the simulation RF data.

The learner 131 is structured to include a plurality of layers each including a plurality of units U. Normally, the learner 131 is structured to include an input layer, an output layer, and an intermediate layer. The input layer is positioned on the side closest to the input. The output layer is positioned on the side closest to the output. The intermediate layer is provided between the input layer and the output layer. There is one intermediate layer in the example in FIG. 7; however, there may be a plurality of intermediate layers.

Each input variable is input to each unit U of the input layer. In each unit U, weights w₁, w₂, w₃, . . . for the respective input variables and a bias b are defined. The value obtained by adding the bias to the sum of values each of which is obtained by multiplying a corresponding one of the input variables by its corresponding weight is an input u of the unit U.

Each unit U outputs an output f(u) of a function f called an activation function with respect to the input u. As the activation function, for example, a sigmoid function, a ramp function, a step function, or the like can be used. An output from each unit U of the input layer is input to each unit of the intermediate layer. That is, all of the units U of the input layer and all of the units of the intermediate layer are connected to each other.

Each unit of the intermediate layer receives, as an input, an output from each unit U of the input layer and performs substantially the same processing described above. That is, in each unit of the intermediate layer, weights corresponding to the respective units U of the input layer and a bias are set. An output from each unit of the intermediate layer is input to each unit of the output layer. That is, all of the units of the intermediate layer and all of the units of the output layer are connected to each other.

Each unit of the output layer receives, as an input, an output from each unit of the intermediate layer and performs substantially the same processing described above. That is, in each unit of the output layer, weights corresponding to the respective units of the intermediate layer and a bias are set.

An output from each unit of the output layer is output data from the learner 131. Output variables y₁, y₂, y₃, . . . included in the output data become pixel values of pixels of a reconstructed image.

The training processing unit 134 adjusts the weights and bias of each unit of each layer such that the values of the output variables in the output data corresponding to the simulation RF data approach pixel values of a measurement image.

The data collection unit 135 collects (including receives or acquires) measurement data (RF data), which are data obtained by the plurality of elements, via the switch circuit 110 and the emission-reception circuit 120. The RF data is stored in the RF data storage area 133 of the memory unit M.

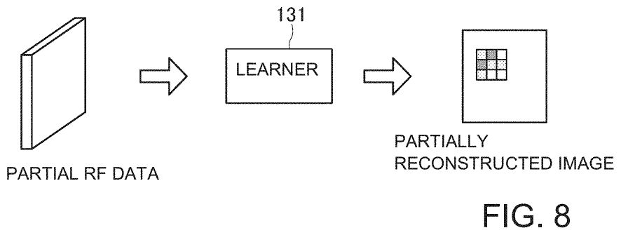

The image acquisition unit 136 extracts, from the RF data, partial RF data corresponding to a point of interest. As illustrated in FIG. 8, the image acquisition unit 136 inputs, as input data, partial RF data to the learner 131, which has been sufficiently trained, and acquires pixel values of a partially reconstructed image output from the learner 131. The image acquisition unit 136 repeatedly performs the processing described above for a plurality of points of interest included in a region of interest (ROI). As a result, a tomographic image corresponding to the ROI of the test object T can be reconstructed. The reconstructed image is displayed on the image display device 140. In particular, in the embodiments of the present invention, a data generation time for learning can be greatly improved by setting an enormous number of points of interest on one image. For example, in a case where the number of pixels is N² and the distance between adjacent pixels for which the independence of information for each pixel is not guaranteed is n, N²/n² points of interest can be set as an order and the temporal cost and data size required for the numerical simulation can be significantly reduced.

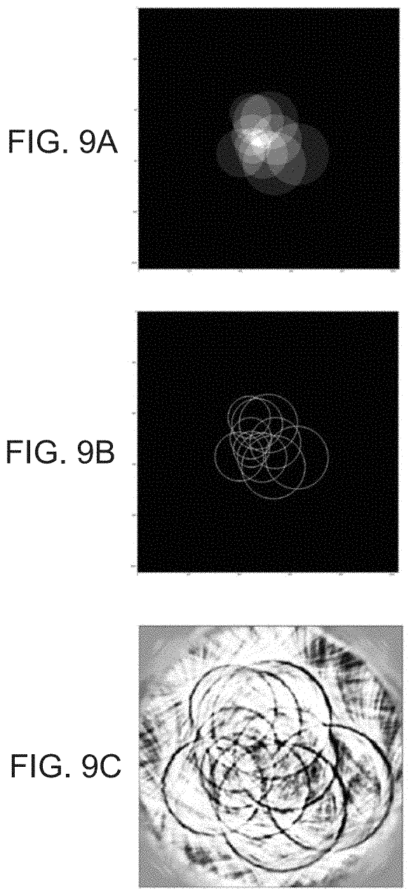

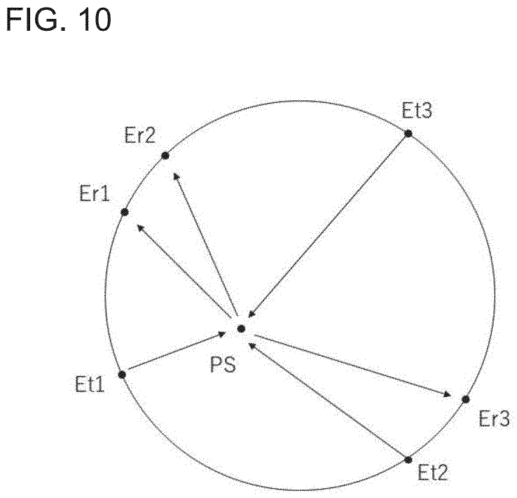

FIG. 9A illustrates an example of a biological model, which is a material density distribution. FIG. 9B illustrates a measurement image of this biological model. FIG. 9C illustrates an example of a reconstructed image obtained from output data by inputting simulation RF data for this biological model to the learner 131, which has already been trained. It was confirmed that it was possible to obtain a clear reconstructed image.

In this manner, according to the present embodiment, since RF data (partial RF data) is simply input to the learner 131 without performing an approximation calculation or the like thereon, accurate image reconstruction can be performed without reducing the amount of information of the RF data. Moreover, according to the present embodiment, training data generated through a simulation can be used to train the learner 131. Here, in a case where a clinical image is used as training data, a huge number of clinical images with doctors' diagnosis results are generally required in order to ensure the accuracy of learning. However, it is not easy to collect a necessary and sufficient number of clinical images. Moreover, as a result of using only data for which diagnoses are made by human beings, images outside the range of existing image data cannot be learned, and thus it is difficult to completely eliminate sample bias of data to be used. Therefore, use of training data generated through a simulation can suppress the generation cost of training data and reduce the training-data bias and variation in accuracy.

The memory unit M may further store a second learner. The second learner learns using training data including tomographic images (for example, past medical images) and tumor information determining the positions of tumors included in the tomographic images and can determine the presence or absence and position of a tumor in tomographic images of a test object.

The calculation device 130 further includes a determination unit. The determination unit inputs a tomographic image acquired by the image acquisition unit 136 to the second learner and outputs, on the basis of output data from the second learner, a determination result on the presence or absence of a tumor in the tomographic image and, in a case where a tumor exists, information on the position of the tumor.

Partial RF data to be input to the learner 131 may include not only RF data corresponding to the curved surface C as illustrated in FIG. 6 but also RF data of an area surrounding the curved surface C.

When the number of emission conditions N, the number of reception elements M, and the number of sample points n in the time direction are used, the size of partial RF data illustrated in FIG. 6 is N×M×n. In the description made so far, which value to which n is set has not been described in detail. Under conditions where the effect caused by the heterogeneity of sound speed is small, n=1 or n can be set to a value close to 1. In contrast, under conditions where the effect caused by the heterogeneity of sound speed is large, uncertainty increases when the distances illustrated in FIG. 5 are converted into propagation times. Thus, it is preferable that n be set to a relatively large value. For example, a value is obtained by dividing the difference between a propagation time when the sound speed is fastest and a propagation time when the sound speed is slowest in a medium that may be present in the paths by a sampling period, and n is set to a value obtained by multiplying the value by the dispersion in propagation time. As an effect obtained through learning using the structure according to the present embodiment, the heterogeneity of sound speed can be expected to be corrected by setting n in this manner.

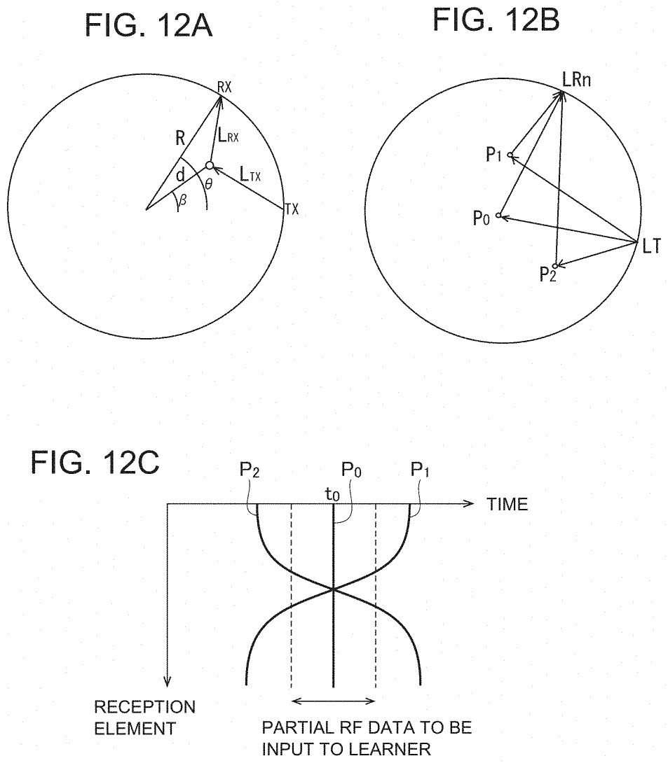

FIG. 12A is a diagram illustrating an example of ultrasound propagation paths. When reflected waves that are waves emitted from an emission point TX and reflected by each scatter point PI are received at a reception point RX, a propagation time from the emission point TX to the reception point RX can be obtained by the following Equation 1, in which the radius of the area of a circle surrounded by the ring array is R, the distance between the center of the circle and the scatter point PI is d, the distance from the emission point TX to the scatter point PI is L_{TX}, the distance from the scatter point PI to the reception point RX is L_{RX}, the position of the emission point TX is (R, ω), and the position of the reception point is (R, θ).

L_{TX}=\sqrt{R²+d²−2Rd cos[ω−β]}

L_{RX}=\sqrt{R²+d²−2Rd cos[θ−β]} (1)

Since the propagation time is proportional to

t∝L_{TwoWay}=L_{TX}+L_{RX} (2)

distance, a propagation time at a certain time t can be expressed as in the following Equation 2.

FIG. 12B is a diagram illustrating an example of ultrasound propagation paths in a case where reflected waves that are waves emitted from an emission point LT and reflected by point scatterers P₀, P₁, and P₂ are received at a reception point LRn. FIG. 12C illustrates RF data in a case where the emission point LT is used. The vertical axis represents reception-element number, and the horizontal axis is a time axis (reflected-wave propagation time).

For example, in a case where the position of the point scatterer P₀ is to be determined, RF data at a time to includes not only a reflected-wave component from the point scatterer P₀ but also reflected-wave components from the point scatterers P₁ and P₂. Thus, preferably, partial RF data to be input to the learner 131 is not data only at the time to but is data having a width to some extent and including the time to (the number of sample points n in the time direction is increased). The RF data that each scatter point has includes information unique to the scatter point. For example, the RF data has a unique trajectory based on the position of the scatter point and has a signal strength that varies depending on the scattering intensity dependent on the characteristics of living tissue that the scatter point has. Thus, by using information that is continuous over time, it becomes easier to detect effects due to the reflected-wave components from characteristics of the RF data of each of the point scatters P₁ and P₂. Consequently, the image quality of a partially reconstructed image corresponding to the point scatterer P₀, which is a point scatterer of interest, can be improved. It is difficult to distinguish partial RF data to be input to the learner 131 from other RF data if the temporal width of the partial RF data is too small, and the man-hour cost for deep learning increases if the temporal width is too large. Therefore, preferably, a width is selected that sufficiently satisfies both conditions.

Next, another embodiment of the present invention will be described, the other embodiment being for another application other than correction of the heterogeneity of sound speed. In imaging using a ring array, when the number of emission conditions is increased, the imaging time increases and the size of acquired RF data increases. In RF data for which an appropriate sampling frequency is set with respect to the center frequency, in a case where the number of emission conditions is on the order of 100 and the number of reception elements is on the order of 1000, the data size per cross section is on the order of a few hundred GB. The amount of three-dimensional data for an entire breast ends up being a significantly massive amount of data as large as 1 TB.

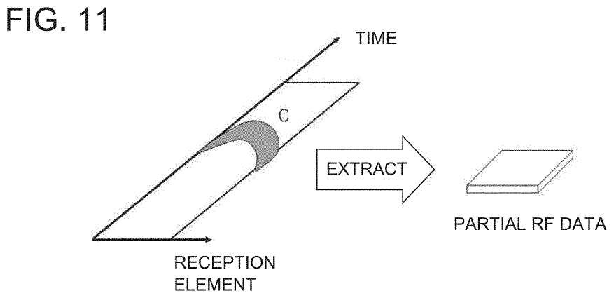

In contrast, in order not to create a region where the signal-to-noise ratio decreases, it is effective to introduce sound energy into the entire imaging region through provision of ultrasonic energy from multiple directions. In an existing synthetic aperture method, a propagation time is obtained by determining which emission point (Et), which scatter point (PS), and which reception point (Er) are to be used, so that imaging can be performed. However, according to a method according to the present invention, when emission is performed simultaneously from a plurality of emission points Et (the drawing illustrates an example of three emission points Et) and reception is performed at each Er as illustrated in FIG. 10, a strip-shaped region is a region where corresponding data is stored as illustrated in FIG. 11. (As a matter of course, the data in this strip is not constituted only by the corresponding data).

The line C is spread out into a strip-shaped region in an existing synthetic aperture method, thereby resulting in a blurred image. In contrast, in the present embodiment, by extracting partial RF data from this strip-shaped data and learning the partial RF data, it becomes possible to perform imaging while suppressing blurring and suppressing inconsistencies in the signal-to-noise ratio inside the entire imaging region due to the small number of emission conditions. As a result of this, the imaging time can be reduced and the total data amount of RF data can be reduced.

Note that, in the embodiments of the present invention, a simulation calculation for ultrasound propagating through a biological model can be performed by solving a wave equation using the finite-difference time-domain method or by performing a calculation using a method such as ray tracing. The biological model is obtained by discretizing a space and setting, for example, a sound speed, a density, and an attenuation factor at each discrete point. Here, scattering of ultrasound is given by the spatial gradient of acoustic impedance, which is the product of the sound speed and the density, and thus the attenuation factor is given as disturbance similarly to the heterogeneity of sound speed. To perform robust learning for such disturbance is also an advantage of learning using various biological models.

To train the learner 131, a brightness image, which is a gray scale image into which a natural image such as an image of an animal or a landscape is converted, may be used as training data instead of a biological model using a simulation. This is because a natural image includes various spatial frequency components and thus provides an environment more similar to that in a living body that is an imaging target in a medical image. When random pattern images are artificially generated to construct a biological model for training, higher spatial frequency components are relatively more likely to be present than lower spatial frequency components, so that there are differences between features of the biological model and features that should be learned in training as clinical images. If a large number of clinical images are available, this is appropriate for the objective; however, it is generally difficult to collect a large number of clinical images, and clinical images do not have sufficiently high spatial frequencies because the spatial resolution of clinical images is limited. Therefore, clinical images are not always appropriate as a training model. Use of natural images is advantageous in terms of the two points described above.

In this case, first, a brightness image is converted into an acoustic feature distribution image. Note that, in the present embodiment, an example will be described in which a density distribution image is used as an acoustic feature distribution image; however, for example, a sound speed distribution image or the like can also be applied. The following equation can be used to perform image conversion. In the following equation, σ₁(x, y) is a density corresponding to a pixel (x, y), σ₀ is a reference density, I(x, y) is a brightness (pixel value) of [0, 1] at the pixel (x, y) of the brightness image, σₘₐₓ is a maximum amplitude, and ε is a random number of [−1, 1]. σ₁(x, y)=σ₀+I(x, y)*σₘₐₓ*ε

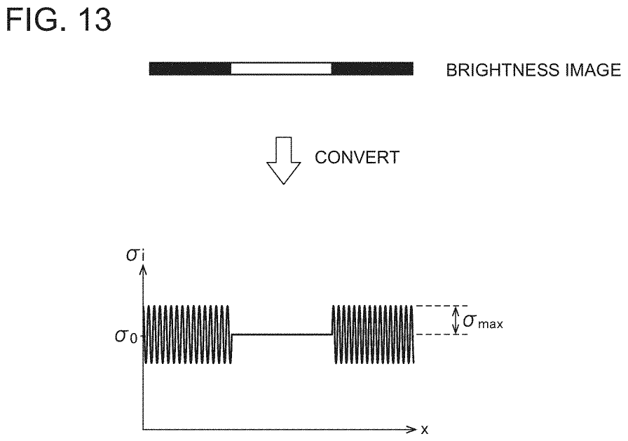

FIG. 13 illustrates an example in which a pixel sequence (a brightness image) is converted into a density distribution. In the pixel sequence, a black portion, a white portion, and a black portion are arranged in order.

In a simulation space, the size and arrangement of the plurality of elements E of the ring array R are simulated for the density distribution, and RF data (measurement data) of a simulation is input to the learner 131. In the simulation, reflected ultrasound from the density distribution is received by a plurality of elements while switching an emission element. The learner 131 is trained such that output data from the learner 131 match pixel values of the brightness image.

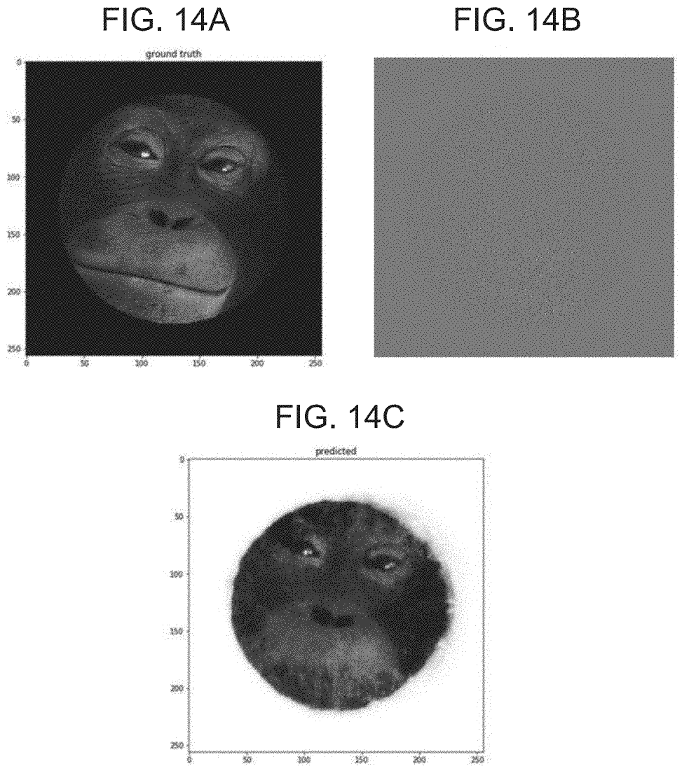

FIG. 14A illustrates a brightness image that is a gray scale image into which an image of a chimpanzee is converted. FIG. 14B illustrates a density distribution image generated from the brightness image of FIG. 14A. FIG. 14C illustrates a reconstructed image obtained from output data from the learner 131, which has already been trained and into which simulation RF data for the density distribution of FIG. 14B is input. It was confirmed that it was possible to obtain a clear reconstructed image.

In the embodiment described above, the configuration using a ring array has been described; however, a probe may also be used on which elements that emit and receive ultrasound are arranged in a straight line or a plane.

According to the embodiments of the present invention, a clear, accurate ultrasound image can be generated.

The present invention has been described in details using specific embodiments; however, it is obvious to those skilled in the art that various changes can be made without departing from the gist and scope of the present invention.

What is claimed is:

1. An ultrasound diagnostic system comprising:
a plurality of elements that perform at least one of emission of ultrasound to a test object and reception of reflected ultrasound that is ultrasound reflected from the test object;
a data collection unit that collects measurement data of the reflected ultrasound through at least one of the plurality of elements while switching an element that emits ultrasound; and
a first learner that learns using training data including a test-object model and simulation measurement data to output a tomographic image of the test object from the measurement data, which is input to the first learner, the test-object model being expressed by a distribution of acoustic features, the simulation measurement data being obtained by emitting ultrasound while switching an element that emits ultrasound and receiving reflected ultrasound from the test-object model using at least one of the plurality of elements in a simulation space in which a size and an arrangement of the plurality of elements are simulated.

2. An ultrasound diagnostic system comprising:
a plurality of elements that perform at least one of emission of ultrasound to a test object and reception of reflected ultrasound that is ultrasound reflected from the test object;
a data collection unit that collects measurement data of the reflected ultrasound through at least one of the plurality of elements while switching an element that emits ultrasound; and
a first learner that learns using training data including a brightness image and simulation measurement data to output a tomographic image of the test object from the measurement data, which is input to the first learner, the brightness image being based on a natural image, the simulation measurement data being obtained by emitting ultrasound while switching an element that emits ultrasound and receiving reflected ultrasound from an acoustic feature distribution based on the brightness image using at least one of the plurality of elements in a simulation space in which a size and an arrangement of the plurality of elements are simulated.

3. The ultrasound diagnostic system according to claim 1, further comprising: an image acquisition unit that inputs the measurement data collected by the data collection unit to the first learner and acquires a tomographic image output from the first learner.

4. The ultrasound diagnostic system according to claim 2, further comprising: an image acquisition unit that inputs the measurement data collected by the data collection unit to the first learner and acquires a tomographic image output from the first learner.

5. The ultrasound diagnostic system according to claim 3, further comprising:
a second learner capable of determining a presence or absence of and a position of a tumor in a tomographic image of a test object through learning using training data including a training tomographic image and tumor information determining a position of a tumor included in the training tomographic image; and
a determination unit that inputs the tomographic image acquired by the image acquisition unit to the second learner and outputs, on a basis of output data from the second learner, a determination result on a presence or absence of a tumor in the tomographic image and, in a case where a tumor exists, information on a position of the tumor.

6. The ultrasound diagnostic system according to claim 4, further comprising:
a second learner capable of determining a presence or absence of and a position of a tumor in a tomographic image of a test object through learning using training data including a training tomographic image and tumor information determining a position of a tumor included in the training tomographic image; and
a determination unit that inputs the tomographic image acquired by the image acquisition unit to the second learner and outputs, on a basis of output data from the second learner, a determination result on a presence or absence of a tumor in the tomographic image and, in a case where a tumor exists, information on a position of the tumor.

7. The ultrasound diagnostic system according to claim 1, wherein the plurality of elements are arranged around the test object.

8. The ultrasound diagnostic system according to claim 2, wherein the plurality of elements are arranged around the test object.

9. The ultrasound diagnostic system according to claim 3, wherein the plurality of elements are arranged around the test object.

10. The ultrasound diagnostic system according to claim 4, wherein the plurality of elements are arranged around the test object.

11. The ultrasound diagnostic system according to claim 5, wherein the plurality of elements are arranged around the test object.

12. The ultrasound diagnostic system according to claim 6, wherein the plurality of elements are arranged around the test object.
